(19) United States
(12) Patent Application Publication
(10) Pub. No.: US 2024/0309445 A1
(43) Pub. Date: Sep. 19, 2024

(54) METHODS FOR SEQUENCING NUCLEIC ACID MOLECULES WITH SEQUENTIAL BARCODES

(71) Applicant: Ultima Genomics, Inc., Newark, CA (US)

(72) Inventors: Florian OBERSTRASS, Redwood City, CA (US); Gilad ALMOGY, Palo Alto, CA (US)

(21) Appl. No.: 18/281,930

(22) PCT Filed: Mar. 22, 2022
(86) PCT No.: PCT/US2022/071272

§ 371 Date: Sep. 13, 2023

Related U.S. Application Data
(60) Provisional application No. 63/164,958, filed on Mar. 23, 2021.

(51) Int. Cl. C12Q 1/6874; C12N 15/10

(52) U.S. Cl. CPC C12Q 1/6874; C12N 15/1065

(57) ABSTRACT

Methods for determining a sequence of a polynucleotide comprising two or more barcode regions and an intervening region thereof are described herein. Flow sequencing methods can be used to sequence the barcode regions by extending a sequencing primer in a plurality of discrete flow steps, which include combining a primer/polynucleotide hybrid with a nucleotide that is incorporated into the extending primer if a complementary base in the poly nucleotide is present at the primer terminus. For one or more regions in the polynucleotide (e.g., barcode regions, a region of interest in the polynucleotide), the presence or absence of an incorporated nucleotide is detected (e.g., the sequence is determined for the said regions). For one or more regions in the polynucleotide (e.g., intervening regions), the primer can be extended without detecting the presence or absence of an incorporated nucleotide (e.g., the sequence is not determined), thereby increasing efficiency of the primer extension.

### CROSS-REFERENCE TO RELATED APPLICATION

This application claims the priority benefit of U.S. Provisional Patent Application Ser. No. 63/164,958, filed Mar. 23, 2021; the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165272000940SEQLIST.TXT, date recorded: Mar. 22, 2022, size: 1,571 bytes).

### FIELD OF THE INVENTION

Described herein are methods of sequencing a polynucleotide with two or more barcode regions.

### BACKGROUND

Massively parallel sequencing, also referred to as next-generation sequencing (NGS), often includes pooling polynucleotides from several different origins. Sequencing barcodes, synthetic polynucleotide sequences, can be attached (either directly, such as by ligation, or indirectly, such as by amplification (e.g., PCR) or reverse transcription) to polynucleotides prior to sequencing. Polynucleotides from the same origin can be labeled with the same barcode, which allows tracing of the polynucleotides to the polynucleotide origin. The barcodes are sequenced with the polynucleotides of interests such that the resulting sequencing read has the barcode associate with the sequencing read, thereby associating the sequencing read with the polynucleotide origin.

Single-cell sequencing technologies allow the origin of a polynucleotide to be traced to single cell. Polynucleotides from the same cell can be labeled with the same barcode or combination of barcodes so that when the polynucleotides in from many different cells are sequenced in parallel, the barcode or combination of barcodes allows the sequenced polynucleotides to be clustered according to the cell of origin. See, for example, the split-pool ligation based transcriptome sequencing (SPLiT-seq) method described in Rosenberg et al., Single-cell profiling of the developing mouse brain and spinal cord with split-pool barcoding, Science, vol. 360, pp. 176-182 (2018).

Certain barcoding strategies, such as some single-cell sequencing strategies, include the use of two or more barcode regions that are sequentially attached. The sequential attachment can, in some instances, result in an intervening region between the barcodes. While the information for the barcodes in necessary to trace the origin of the polynucleotide of interest, the intervening region is generally unimportant. Sequencing unimportant regions wastes time and increases sequencing costs. Further, the combination of barcodes and intervening regions causes a lengthy sequencing distance before the polynucleotide of interest is sequenced, which, for many sequencing methods, can result in degraded sequencing quality.

### BRIEF SUMMARY OF THE INVENTION

Methods for determining a sequence of a polynucleotide comprising two or more barcode regions on the same end of the polynucleotide relative to a sequence of interest (e.g., a region of interest in a polynucleotide) are described herein. For example, a method of determining a sequence of a polynucleotide comprising two or more barcode regions can include hybridizing a primer to the polynucleotide to form a hybrid, wherein the polynucleotide comprises a first barcode region, a second barcode region, and an intervening region between the first barcode region and the second barcode region; sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark sequencing flow steps comprising combining the hybrid with nucleotides without detecting the presence or absence of an incorporated nucleotide; and sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide. The method may further include associating the two or more barcode regions with the region of interest.

The intervening region has a known sequence (i.e., known at the start of the method). If the sequence is known, the set of one or more dark sequencing flow steps used to extend the primer through the intervening region can be configured to extend the primer during each dark sequencing flow step. The intervening region may be formed, for example, by ligation or PCR amplification, which can allow the intervening region to be known.

The method can include sequencing the region of interest in the polynucleotide. The region of interest may be sequenced after sequencing the first barcode region and the second barcode region.

The method can further include extending the primer through a second intervening region using a second set of one or more dark flow steps comprising combining the hybrid with nucleotides without detecting the presence or absence of an incorporated nucleotide; and sequencing a third barcode region in the polynucleotide using a third plurality of sequencing flow steps comprising combining the hybrid with labeled nucleotides and detecting the presence or absence of an incorporated nucleotide; wherein the second intervening region is between the second barcode region and the third barcode region, and wherein the third barcode region is on the same end of the polynucleotide relative to the sequence of interest as the first barcode region and the second barcode region.

The region of interest may be sequenced after sequencing the first barcode region, the second barcode region, and the third barcode region. The second the second intervening region can have a known sequence. By knowing the sequence of the second intervening region, the second set of one or more dark sequencing flow steps used to extend the primer through the second intervening region can be configured to extend the primer during each dark sequencing flow step. The second intervening region may be formed, for example, by ligation or PCR amplification, which can allow the intervening region to be known.

The nucleotides used in the first plurality of sequencing flow steps or the nucleotides used in the second plurality of sequencing flow steps may include non-terminating nucleotides. The nucleotides used in the set of one or more dark sequencing flow steps may include non-terminating nucleotides. The nucleotides used in the first plurality of sequencing flow steps or the nucleotides used in the second plurality of sequencing flow steps may include labeled nucleotides and unlabeled nucleotides. The nucleotides used in the dark sequencing flow steps may include unlabeled nucleotides. The nucleotides used in the dark sequencing flow steps comprise only unlabeled nucleotides.

The polynucleotide can optionally further include a unique molecular identifier. The unique molecular identifier may be directly fused to one of the two or more barcode regions, or may be separated by an intervening region.

The nucleotides used in each sequencing flow step of the first plurality of sequencing flow steps may include a single type of nucleotide base. Alternatively, the nucleotides used in at least a portion of the flow steps of the first plurality of sequencing flow steps can include two or three different types of nucleotide bases.

The nucleotides used in each sequencing flow step of the second plurality of sequencing flow steps can include a single type of nucleotide base. Alternatively, the nucleotides used in at least a portion of the flow steps of the second plurality of sequencing flow steps can include two or three different types of nucleotide bases.

The nucleotides used in each dark sequencing flow step can include a single type of nucleotide base. Alternatively, the nucleotides used in at least a portion of the dark sequencing flow steps can include two or three different types of nucleotide bases.

The two or more barcode regions, in combination, can uniquely identify a cell of origin for the polynucleotide. For example, the method may further include labeling a nucleic acid molecule from a cell with a cell-specific combination of the two or more barcode regions to form the polynucleotide. The two or more barcode regions can include three or more barcode regions on the same end of the polynucleotide relative to the sequence of interest, each barcode region separated from an adjacent barcode region by an intervening region. Labeling the nucleic acid molecule can include, for example, ligating the at least one of the two or more barcode regions to the nucleic acid molecule. Labeling the nucleic acid molecule can include ligating the at least one of the two or more barcode regions to the nucleic acid molecule within a cell. Labeling the nucleic acid molecule can include reverse transcribing an mRNA molecule to form a cDNA molecule comprising one of the two or more barcode regions. The mRNA molecule may be reverse transcribed within the cell. Labeling the nucleic acid molecule can include attaching at least one of the two or more barcode regions to the polynucleotide by polymerase chain reaction (PCR) amplification. For example, labeling the nucleic acid molecule can include attaching at least one of the two or more barcode regions to the polynucleotide by polymerase chain reaction (PCR) amplification outside of the cell.

The method may be employed such that a sequence of each of a plurality of polynucleotides having different sequences is determined in parallel. At least a portion the polynucleotides in the plurality of polynucleotides may have different sequences have the same two or more barcode regions. Polynucleotides having the same two or more barcode regions may be associated with the same cell of origin.

### BRIEF DESCRIPTION OF THE DRAWINGS

FIG. 1 shows an exemplary polynucleotide that may be sequenced according to the methods described herein, in accordance with some embodiments.

FIG. 2A shows an exemplary polynucleotide that may be sequenced according to the methods described herein, in accordance with some embodiments.

FIG. 2B shows an exemplary polynucleotide that may be sequenced according to the methods described herein, in accordance with some embodiments.

FIG. 3 shows an exemplary method for labeling polynucleotides with multiple barcode regions, in accordance with some embodiments.

FIG. 4 illustrates example polynucleotides, in accordance with some embodiments.

### DETAILED DESCRIPTION OF THE INVENTION

Methods for determining a sequence of a polynucleotide comprising two or more barcode regions and an intervening region between the barcode regions are described herein. The barcode regions are on the same end of the polynucleotide relative to a sequence of interest. Flow sequencing methods, as further described herein, can be used to sequence the barcode regions. The flow sequencing methods can include extending a sequencing primer in a plurality of discrete flow steps, which include combining a primer/polynucleotide hybrid with a nucleotide (e.g., a non-terminating nucleotide) that is incorporated into the extending primer if a complementary base in the polynucleotide is present at the primer terminus. If the flow step is a “read” or “bright” flow step, the presence or absence of an incorporated nucleotide is detected. If the flow step is a “no read” or “dark” flow step, the primer can be extended without detecting the presence or absence of an incorporated nucleotide, thus increasing efficiency of the primer extension. Thus, the sequence of barcode regions can be determined using bright flow steps and the primer can be extended through unimportant regions, such as an intervening region between two barcodes, using dark flow steps.

For example, a method of determining a sequence of a polynucleotide comprising two or more barcode regions can include: hybridizing a primer to the polynucleotide, comprising a first barcode region, a second barcode region, and an intervening region between the first barcode region and the second barcode region, to form a hybrid; sequencing the first barcode region in the polynucleotide using a first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark sequencing flow steps comprising combining the hybrid with nucleotides without detecting the presence or absence of an incorporated nucleotide; and sequencing the second barcode region in the polynucleotide using a second plurality of sequencing flow steps comprising combining the hybrid with nucleotides, wherein at least a portion of the nucleotides are labeled, and detecting the presence or absence of an incorporated nucleotide.

The sequencing process can be applied to polynucleotides containing more than two barcode regions. For example, the flow sequencing method can alternate between “bright” and “dark” flow step to sequence two, three, four, five, or more barcode regions and any intervening region, or portion thereof, between the barcode regions.

The intervening region may be known (i.e., pre-defined) prior to sequencing and can be independent of the sequence of the barcode region. For example, the same known sequence may be introduced by ligation, amplification, reverse transcription, or other attachment method when a subsequence barcode region is attached to a polynucleotide across a plurality of polynucleotides sequenced in parallel. Knowing the intervening region is particularly advantageous because the set of dark sequencing flow steps used to extend the primer through the intervening region may be specifically designed to optimize speed of primer extension. For example, the set of one or more dark sequencing flow steps used to extend the primer through the intervening region may be configured to extend the primer during each dark sequencing flow step.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The terms “individual,” “patient,” and “subject” may be used synonymously herein. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base containing solution) to a sequencing reaction space at a first time point and (ii) providing a nucleotide-containing reagent (e.g., a G-base containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), e.g., 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space: (e.g., [A-T-G-C-A-T-G-C-A-T-G-A-T-G-A-T-G-A-T-G-C-A-T-G-C]).

Such a one-dimensional matrix or linear array of bases in the flow order may also be referred to herein as a “flow space.” Each entry in flow space (e.g., each element in the one-dimensional matrix or linear array) may be referred to as a flow position. A flow order may have any number of nucleotide flows. A “flow position,” as used herein, generally refers to the sequential position of a given nucleotide flow in the flow space. A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A-T-G-C], [A-A-T-T-G-G-C-C], [A-T], [A/T-A/G], [A-A], [A], [A-T-G], etc.). A flow cycle may have any number of nucleotide flows. A given flow cycle may be repeated one or more times in the flow cycle, consecutively or non-consecutively. Accordingly, the term “flow cycle order,” as used herein, generally refers to an ordering of flow cycles within the flow order, and can be expressed in units of flow cycles. For example, where [A-T-G-C] is identified as a 1st flow cycle, and [A T G] is identified as a 2nd flow cycle, the flow order of [A-T-G-C-A-T-G-C-A-T-G-A-T-G-A-T-G-A-T-G-C-A-T-G-C] may be described as having a flow-cycle order of [1st flow cycle; 1st flow cycle; 2nd flow cycle; 2nd flow cycle; 2nd flow cycle; 1st flow cycle; 1st flow cycle]. Alternatively or in addition, the flow-cycle order may be described as [cycle 1, cycle, 2, cycle 3, cycle 4, cycle 5, cycle 6], where cycle 1 would be the 1st flow order, cycle 2 would be the 1st flow order, cycle 3 would be the 2nd flow cycle order, etc.

A “dark flow step” or “dark sequencing flow step” refers to a nucleotide flow wherein the presence or absence of an incorporated nucleotide is not detected during the flow step. Nucleotides provided to a target polynucleotide in a dark sequencing flow step may be labeled or unlabeled.

The term “label,” as used herein, refers to a detectable moiety that is coupled to or may be coupled to another moiety, for example, a nucleotide or nucleotide analog. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease). In some embodiments, the label is a fluorophore.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

A “non-terminating nucleotide” is a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

The term “sequencing,” as used herein, generally refers to a process for generating or identifying a sequence of a biological molecule, such as a nucleic acid. The sequence may be a nucleic acid sequence which comprises a sequence of nucleic acid bases.

As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

“Expected sequencing data” refers to sequencing data one would expect if the sequence of a polynucleotide used to generate a coupled sequencing read pair, or the sequence of a region of said polynucleotide, matches a reference sequence. That is, expected sequencing data refers to sequencing results for a subject that do not deviate from a reference sequence.

The term “reference genome,” as used herein, refers to a standardized genomic sequence or a portion thereof (e.g., any genome known in the art). A reference genome may be a representative example of a set of genes. In some instances, a reference genome is generalized to a species (e.g., Homo sapiens) and is determined from one or more assembled or partially assembled genome sequences of one or more individuals of said species. In some instances, a reference genome is specific to an individual of a species, and is such instances the reference genome may be determined from one or more assembled or partially assembled genome sequences from said individual. A reference genome may be any portion of a genomic nucleic acid sequence (e.g., a targeted panel of genes, one or more chromosomes, an entire genome of a species, etc.) that is used as a comparison for generated nucleic acid sequencing data (e.g., sequencing information generated according to sequencing methods described herein). Examples of human reference genomes include NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). Additional reference genomes can be found online in the National Center for Biotechnology Information (NCBI) of the University of California, Santa Cruz (UCSC) genome browsers.

A “short genetic variant” is used herein to describe a genetic polymorph (i.e., mutation) that is 10 consecutive bases in length or less (i.e., 10, 9, 8, 7, 6, 5, 4, 3, 2, or 1 base(s) in length). The term includes single nucleotide polymorphisms (SNPs), multi-nucleotide polymorphisms (MNPs), and insertions or deletions that are 10 consecutive bases in length or less.

The term “terminator,” as used herein with respect to a nucleotide, may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator.

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes: some blocks are, optionally, combined; the order of some blocks is, optionally, changed; and some blocks are, optionally, omitted. In some examples, additional steps (e.g., blocks) may be performed in combination with the exemplary processes. Accordingly, the operations as illustrated (and as described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Sequenced Polynucleotides

Polynucleotides sequenced according to the methods described herein include a region of interest and two or more barcode regions on the same side of the polynucleotide relative to the region of interest. For example, the two or more barcode regions may be on the 3′ end of the polynucleotide relative to the region of interest or on the 5′ end of the polynucleotide relative to the sequence of interest. The position of the barcode sequence relative to the region of interest can depend on the method used to label the polynucleotide with the two or more barcode regions. The two or more barcode regions are separated by an intervening region, which may have a known sequence. The two or more barcode regions can include two, three, four, five, six, or more barcode regions, each of which may be separated by an intervening region (which may be known prior to sequencing the polynucleotide) and on the same end of the polynucleotide relative to the region of interest.

The sequence (e.g., region) of interest is the targeted nucleic acid molecule whose sequence is desired. The sequence of interest may also be referred to as an “insert”. In massively parallel sequencing methods, many different regions of interest are sequenced simultaneously, and barcode regions (e.g., barcode regions attached to the target nucleic acid molecule) can be used to trace the origin of any given sequence to an original nucleic acid molecule. Thus, the barcodes (i.e., a combination of barcodes) can be associated with the region of interest.

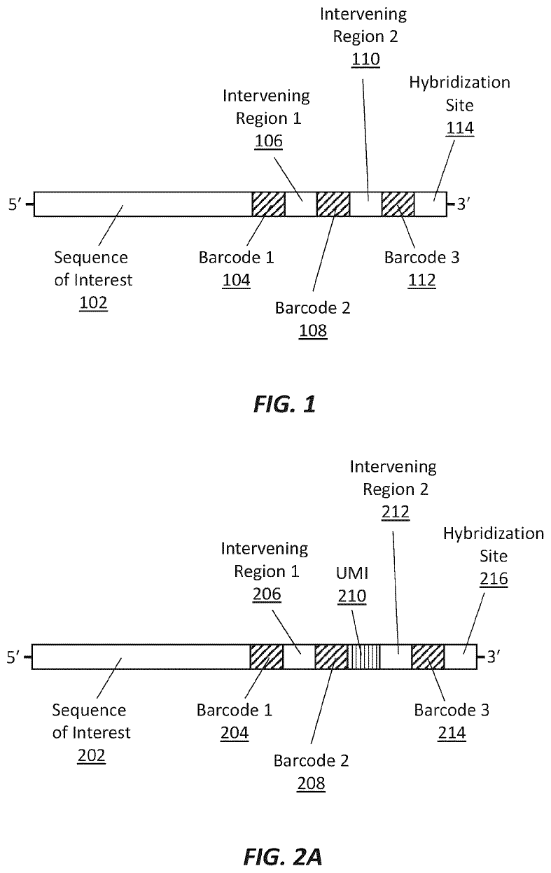

FIG. 1 illustrates an exemplary polynucleotide that may be sequenced according to the methods described herein. The exemplary polynucleotide shown in FIG. 1 includes, from 5′ to 3′, a sequence of interest 102 (e.g., a region of interest), a first barcode region 104, a first intervening region 106, a second barcode region 108, a second intervening region 110, a third barcode region 112, and a hybridization site 114. Although the illustrated polynucleotide includes three barcode regions and two intervening regions, the polynucleotide sequenced according to the methods described herein may include more or fewer barcode regions and intervening regions. For example, the polynucleotide may include two barcode regions and one intervening region, or four barcode regions and three intervening regions, etc. . . . Each barcode region will be separated from another barcode region by an intervening region.

The hybridization site 114 allows a primer (i.e., a sequencing primer) to hybridize to the polynucleotides so that the barcode regions (e.g., 104, 108, 112) and the sequence of interest 102 can be sequenced. Generally, the hybridization site is common to all polynucleotides that are being sequenced in parallel, although this is not required. The barcode regions, and the intervening regions that separate the barcode regions, can be positioned within the polynucleotide so that the barcode regions are between the sequence of interest and the hybridization site. Thus, the sequencing primer can hybridize to the hybridization site and be extended through the barcode regions and intervening regions (which extension may be used to generate sequencing information for the barcode regions) before being extended through some or all of the sequence of interest (which additional extension may be used to generate sequencing information for some or all of the sequence of interest). The hybridization site may be directly fused to the 3′ end of the final barcode region, or an intervening region may be positioned between the final barcode and the hybridization site.

In some instances, two or more barcodes (and one or more corresponding intervening regions) may be disposed 5′ of the sequence of interest. That is, similarly but in reverse of what is illustrated in FIG. 1, the sequencing primer may hybridize to the hybridization site, be extended through some or all of the sequence of interest, and then extended through the barcode regions and intervening region(s).

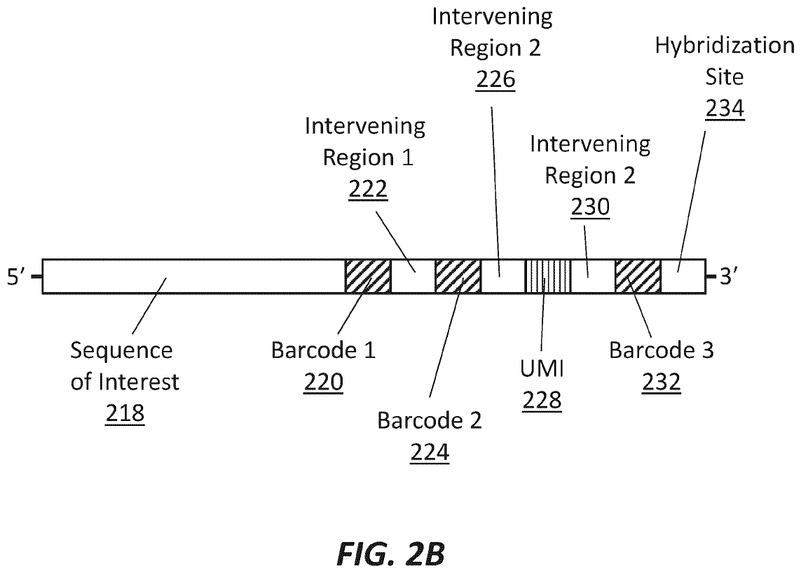

The barcode regions can be used to trace the origin of the region of interest. For example, the barcode regions can provide a unique sequence that associates the sequence of interest with a particular cell, and polynucleotides originating from the same cell can include the same combination of barcode regions. Thus, when a plurality of polynucleotides are sequenced, different sequences associated with the same two or more barcode regions can be traced to the same cell. Optionally, the polynucleotide may include a unique molecular identifier (UMI) sequence, which may be different from other polynucleotides originating from the same cell and can be used to distinguish different polynucleotides. The UMI may be directly fused to one of the two or more barcode regions or may be separated from any of the barcode regions by an intervening region. If labeled polynucleotides are amplified (for example, during the preparation of a sequencing library), the amplified polynucleotides retain the same barcode regions (and, if present, UMI). FIG. 2A illustrates an exemplary polynucleotide with a UMI that may be sequenced according to the methods described herein. The exemplary polynucleotide shown in FIG. 2A includes, from 5′ to 3′, a sequence of interest 202 (e.g., a region of interest), a first barcode region 204, a first intervening region 206, a second barcode region 208, a UMI 210 directly fused to the second barcode region 208, a second intervening region 212, a third barcode region 214, and a hybridization site 216. FIG. 2B illustrates another exemplary polynucleotide with a UMI that may be sequenced according to the methods described herein. The exemplary polynucleotide shown in FIG. 2B includes, from 5′ to 3′, a sequence of interest 218, a first barcode region 220, a first intervening region 222, a second barcode region 224, a second intervening region 226, a UMI 228, a third intervening region 230, a third barcode region 232, and a hybridization site 234. In FIGS. 2A and 2B, the UMI is illustrated 3′ of the second barcode region, although in other embodiments, the UMI, if present, may be 3′ or 5′ of any of the barcode regions. In general, the UMI if is generally positioned between the hybridization site and the sequence of interest.

The polynucleotide may be a DNA molecule derived from a cell. For example, the DNA may be genomic DNA (for example, chromosomal DNA or an amplification product from chromosomal DNA). Polynucleotides derived from RNA from a cell may additionally or alternatively be sequenced using the methods described herein. For example, in some operations of method described herein, the polynucleotides may be a cDNA molecule or an amplification product of a cDNA molecule. RNA from a cell may be reverse transcribed (either in the cell itself or outside of the cell) to form a cDNA molecule. Reverse transcription to form the cDNA polynucleotide may itself introduce one or more of the two or more barcode regions. The polynucleotides used in the methods described herein may be obtained from any suitable biological source, for example a tissue sample, a blood sample, a plasma sample, a saliva sample, a fecal sample, or a urine sample.

Libraries of the polynucleotides may be prepared through known methods. In some embodiments, the polynucleotides may be ligated to an adapter sequence. The adapter sequence may include a hybridization sequence (i.e., hybridization site) that hybridizes to the primer extended during the generated of the coupled sequencing read pair. Library preparation may include attaching the two or barcodes to a polynucleotide including the sequence of interest (i.e., to label the polynucleotide with the two or more barcodes).

The sequencing method may include sequencing a plurality of different polynucleotides in parallel (i.e., through multiplex sequencing) to determine the sequences of the polynucleotides. UMIs may be used to label polynucleotides such that sequences determined from the same original nucleic acid molecule (or example, prior to amplification) can be grouped together, and, if desired, consolidated. The UMIs need not be unique across all polynucleotides sequenced in parallel, as distinct sequences of interest, as determined by sequencing, can be used to distinguish different polynucleotides having the same UMI, as would be understood by one skilled in the art.

The nucleic acid molecules may be labeled with a barcode region using any number of techniques or combination of techniques. Different barcode regions may be attached to the nucleic acid molecule including the sequencing of interest using the same technique or different techniques. For example, a nucleic acid molecule may be labeled with a first barcode region using a first barcode labeling technique, and the nucleic acid molecule may be labeled with a second barcode region using a second barcode labeling technique. In some embodiments, the nucleic acid molecule is labeled by ligating at least one of the two or more barcode regions to the nucleic acid molecule. In some embodiments, the nucleic acid molecule is labeled by reverse transcribing an mRNA molecule to form a cDNA molecule comprising one of the two or more barcode regions to the nucleic acid molecule. The reverse transcription may occur within the cell. In some embodiments, the nucleic acid molecule is labeled by attaching at least one of the two or more barcode regions to the polynucleotide by polymerase chain reaction (PCR) amplification. For example, a PCR primer can include one of the two or more barcode regions and a precursor polynucleotide is amplified, thus forming the polynucleotide with the barcode region attached by PCR amplification. Attachment of the one or more barcode regions by PCR amplification may occur outside of the cell, and may provide the last barcode region attached the polynucleotide.

The intervening region introduced into the polynucleotide by attachment of the barcode regions can have a known sequence. For example, the sequence of the primers used to attach the barcode regions by reverse transcription or PCR amplification, or the sequences that are ligated, may be synthetically designed or known prior to barcode region(s) attachment. As further discussed herein, the known sequence in the intervening region can be used to set the flow order of nucleotides used to sequence the polynucleotide, which can accelerate sequencing.

The two or more barcode regions of a polynucleotide may be used to trace the cell, organelle, or tissue of origin. For example, the sequencing library may be prepared such that different polynucleotides having the same combination of two or more barcode regions can be associated with the same cell of origin. Such methods may be referred to as “single-cell sequencing” methods, and can include single-cell RNA-sequencing (“scRNA-seq”) methods. For example, the sequence of interest may be labeled using a split-pool barcode labeling method. Split-pool barcode labeling is an iterative barcode labeling process wherein a plurality of cells from a biological sample are split into separate groups, nucleic acid molecules from the cells in each group are labeled with a barcode region with each group being associated with a different barcode region, and the groups are pooled. This process is repeated for a desired number of barcodes. Nucleic acid molecules are labeled with the barcode region while in the cell, except for the final barcode labeling which may occur while the target nucleic acid molecules are within the cell or outside of the cell. By repeating this process, nucleic acid molecules within a given cell (e.g., originating from a same cell) are labeled with the same combination of barcode regions. Because the cells in a sample are randomly split into different groups a statistically unique barcode region combination is associated with a given cell. That is, the two or more barcode regions, in combination, uniquely identify a cell of origin for the nucleic acid molecule.

The methods for labeling nucleic acid molecules with two or more barcode regions can be used to label nucleic acid molecules (e.g., RNA molecules) in a cell. In some embodiments the methods can be used to label nucleic acid molecules in an organelle, such as a nucleus or mitochondrion. Cells or organelles may be fixed prior to labeling the nucleic acid molecules with the two or more barcodes.

One of the one or more barcodes may be attached to the nucleic acid molecule in a cell or organelle by reverse transcription. The following refers to cells but may also be applied to other organelles such as nuclei. The cells can be dived into separate groups (e.g., wells of a multiwall plate), and reverse transcription primers that include a barcode region can be used to reverse transcribed mRNA molecules within the cells, thus forming cDNA molecules labeled with the barcode region. Each group is associated with a different barcode region such that all labeled nucleic acid molecules within the same group are labeled with the same barcode from the reverse transcription primer and no two groups have the same barcode region sequence in the reverse transcription primer. The cells in the various groups can then be pooled, with the barcode labeled cDNA molecules remaining within each cell. Because the sequence of the reverse transcription primer may be known, and further may be the same for all polynucleotides except for the barcode region, the sequence 5′ or 3′ of the barcode region is known and may be the same for all polynucleotides.

One or more (e.g., 2, 3, 4, or more) of the two or more barcode regions may be attached to the nucleic acid molecule by ligation. For example, a nucleic acid molecule including a barcode region may be ligated to the polynucleotide. The polynucleotide ligated to the barcode-containing nucleic acid molecule may already have a barcode region from an earlier labeling method (e.g., a reverse transcription labeling method). Ligation may be blunt-end ligation or may use overhangs. The sequences between barcode regions (i.e., intervening regions) may be known when a nucleic acid molecule containing a barcode region is ligated to the polynucleotide. The following refers to cells but may also be applied to other organelles such as nuclei. Cells (which may already include polynucleotides labeled with a barcode region, for example by reverse transcription) may be divided into separate groups (e.g., wells of a multiwall plate). A nucleic acid molecule including a barcode region is ligated to polynucleotides in each group. Each group is associated with a different barcode region such that all labeled nucleic acid molecules within the same group are labeled with the same barcode by the ligation reaction, and no two groups have the same barcode region sequence in the ligated nucleic acid molecule. However, polynucleotides in different cells within the same group may have a different combination of two or more barcode regions if the polynucleotide was previously labeled with a barcode region, although polynucleotides within the same cell should have the same combination of barcode regions. Cells may then be pooled.

One of the one or more barcodes may be attached to the nucleic acid molecule in a cell or organelle by PCR amplification. The following refers to cells but may also be applied to other organelles such as nuclei. The cells can be dived into separate groups (e.g., wells of a multiwall plate), and PCR amplification primers that include a barcode region can be used to attach a barcode region to the polynucleotides while amplifying the polynucleotide. The cells may be lysed prior to PCR amplification. If lysed, the barcode region attached to the polynucleotides may be the final barcode attached. The PCR amplification primer may optionally include a hybridization side for sequencing, although the hybridization site may be added in a separate step, for example by ligating an adapter to the polynucleotide. Each group is associated with a different barcode region of the PCR amplification primer, and labeled polynucleotides within the same group are labeled with the same barcode region. However, polynucleotides within the same group may have a different combination of two or more barcode regions if the polynucleotides were previously labeled with a barcode region.

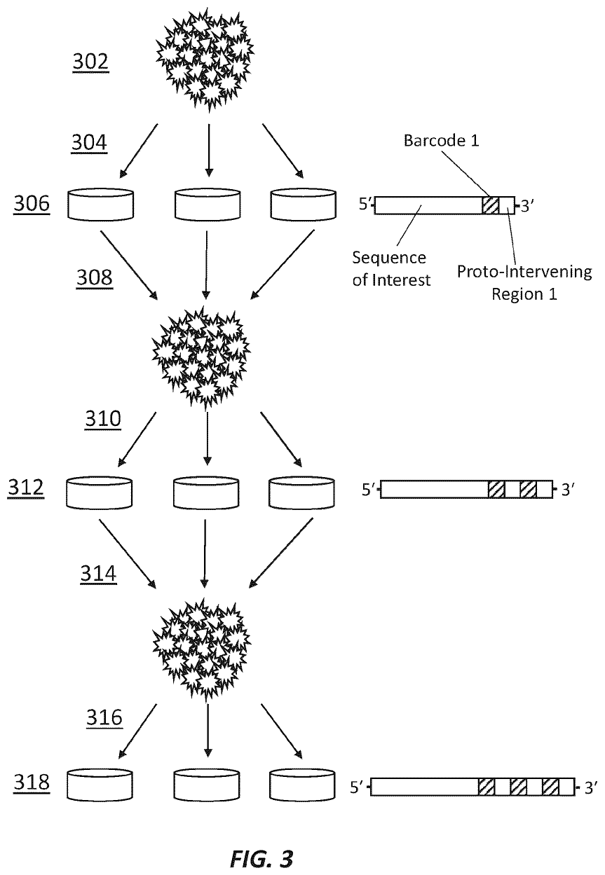

FIG. 3 illustrates an exemplary method for labeling polynucleotides with multiple barcode regions. Cells (or organelles) 302, which may be fixed are split at 304 into a plurality of groups 306, such as wells in a multiwall plate. The cells or organelles are randomly distributed in the different groups. Once in the groups, a reverse transcription primers are added to each group. The reverse transcription primer includes a first barcode region unique for each group such that each group is associated with a different barcode sequence. Optionally, the reverse transcription primer may further include a UMI, which is not the same within a given group. Using the reverse transcription primer, mRNA molecules in the cells are reverse transcribed to form cDNA molecules labeled with the barcode region from the reverse transcription primer. The reverse transcription may introduce a sequence between the barcode region and the sequence of interest, and/or between the barcode region and the terminal end of the cDNA molecule, which is a precursor to the intervening region when the next barcode region is attached to the polynucleotide. Because the sequence of the reverse transcription primer may be known, the sequence of the precursor intervening region, and the sequence of the subsequent intervening region, can be known prior to sequencing. Optionally, the barcode region is at the terminus of the cDNA molecule, and there is no precursor intervening region. Once the cDNA molecules are generated with the first barcode region, the cells from the various groups can be pooled at 308. Pooled cells can gain be randomly divided at 310 into a plurality of groups 312. Once in the groups, nucleic acid molecules comprising a second barcode region associated with a given group are ligated to polynucleotides in the group. Each group is associated with a different second barcode sequence. However, because the cells were randomly divided at 308, any given group will include multiple different combinations of the first and second barcode regions. Further, because polynucleotides were contained within cells, the polynucleotides within a given cell include the same combination of barcode regions. The second barcode region may be directly fused to the first barcode region, which would result in no intervening region. Alternatively, ligation of the nucleic acid molecule that includes the second barcode region to the polynucleotide that includes the first barcode region may result in an intervening region. The intervening region may be due to a terminal sequence in the nucleic acid molecule including the second barcode region, a terminal sequence in the polynucleotide including the first barcode region, or both. The intervening region between the first and second barcode region may be common across all polynucleotides labeled with the two barcode regions. The process of labeling the polynucleotides with barcode regions by ligation may be repeated (i.e. by pooling the cells, dividing the cells, and labeling polynucleotides with a barcode region associate with a particular group) to label the polynucleotide with a third, fourth, fifth, or any number of additional barcode regions. Between any two barcode regions, an intervening region, which may have a known sequence, may be introduced. The cells can then be pooled at 314 and again randomly divided 316 into a plurality of groups. In a final round of barcode labeling (318 as illustrated in FIG. 3), the cells are optionally lysed prior to labeling. At 318, the barcode can be added by PCR amplification using a PCR amplification primer that includes a barcode region. An intervening region between the barcode region attached by PCR amplification and the prior barcode may be introduced by the PCR amplification primer. The PCR amplification primer can have a known sequence, allowing the introduced intervening region sequence to be known. The intervening sequence may be common to all polynucleotides even though the barcode is group-dependent.

Additional methods of labeling nucleic acid molecules in a cell with two or more different barcodes such that the cell of origin of the polynucleotide can be traced are described in Rosenberg et al., Single-cell profiling of the developing mouse brain and spinal cord with split-pool barcoding, Science, vol. 360, pp. 176-182 (2018).

### Flow Sequencing Methods

Sequencing data can be generated using a flow sequencing method that includes extending a primer bound to a template polynucleotide molecule according to a predetermined flow cycle where, in any given flow position, a single type of nucleotide is accessible to the extending primer. In some embodiments, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template polynucleotide molecule. In some embodiments, for example, sequencing data is generated using a flow sequencing method that includes extending a primer using labeled nucleotides, and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473, which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. Further, one or more cycles may omit one or more nucleotides. Solely by way of example, the order of a first cycle may be A-T-G-C and the order of a second cycle may be A-T-C. Alternative orders may be readily contemplated by one skilled in the art. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase 29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Sequencing data, such as a flowgram as described below, can be generated based on the detection of an incorporated nucleotide and the order of nucleotide introduction. For example, a flowgram for the following template sequences is shown in Table 1: CTG and CAG, and a repeating flow cycle of T-A-C-G (that is, sequential addition of T, A, C, and G nucleotides, which would be incorporated into the primer only if a complementary base is present in the template polynucleotide). In Table 1, 1 indicates incorporation of an introduced nucleotide, 0 indicates no incorporation of an introduced nucleotide, and an integer x>1 indicates incorporation of x introduced nucleotides. The flowgram can be used to determine the sequence of the template strand (e.g., the sequence of the template strand may be considered as the complement of the incorporated nucleotides).

TABLE 1

| Flow Cycle | | | | | | | |
|---|---|---|---|---|---|---|---|
| 1 | | | | 2 | | | |
| Cycle Step | | | | | | | |
| 1 | 2 | 3 | 4 | 1 | 2 | 3 | 4 |
| Flow Bases | | | | | | | |
| T | A | C | G | T | A | C | G |

Sequence | Number of Bases Incorporated

| | | | | | | | | |
|---|---|---|---|---|---|---|---|---|
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

A flowgram may be binary or non-binary. A binary flowgram detects the presence (1) or absence (0) of an incorporated nucleotide. A non-binary flowgram, such as shown in Table 1, can more quantitatively determine a number of incorporated nucleotide from each stepwise introduction. A non-binary flowgram also indicates the presence or absence of the base, but can provide additional information including the number of bases incorporated at the given step. For example, the sequence of CCG would incorporate two G bases in one flow cycle step (e.g., in flow cycle 1, cycle step 4), and any signal emitted by the two labeled bases would have a greater intensity than the incorporation of a single base.

Prior to generating the sequencing data, the polynucleotide is hybridized at a hybridization site to a sequencing primer to generate a hybridized template. The polynucleotide may be ligated to an adapter during sequencing library preparation, such as during the attachment of one or more barcode regions to the polynucleotide. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Pat. No. 10,344,328 and International patent application WO 2020/227143, each of which is incorporated herein by reference in its entirety.

The sequencing primer hybridized to the polynucleotide is extended through a region being sequenced, such as a barcode region or a sequence of interest. Intervening regions, such as regions between barcode regions, or between a barcode region and the sequence of interest, are of less interest for sequencing. As discussed above, in some embodiments, the intervening region is known and common to all polynucleotides. Generating sequencing data may be a relatively slow process compared to natural primer extension (with time take to detect nucleotide incorporation and a relatively low per-flow base incorporation rate), and it is generally desirable to increase the speed of extension of the sequencing primer through regions for which it is not necessary or desirable to obtain sequencing data.

To accelerate sequencing of the polynucleotide overall, the primer is extended through the intervening region (e.g., a region not of interest) using one or more acceleration processes. That is, extension of the primer through the intervening region may proceed at a faster extension rate) than the extension of the primer through the barcode regions (or other region(s) of interest). For example, extension of the primer through the intervening region may proceed by extending the primer without detecting the presence or absence of a labeled nucleotide incorporated into the extending primer (i.e., a “no read” or “dark” flow step). During flow sequencing, as discussed above, a labeled nucleotide is incorporated into the extending primer, the hybridized template is washed, and a detector is used to detect a signal from the label of the nucleotide, which indicates whether the nucleotide has been incorporated into the extended primer (which may be referred to as a “read” or “bright” flow step). However, the detection process takes time, and extension of the primer through the intervening region can be accelerated by skipping the detection process. In some embodiments, the primer is extended through the intervening region using unlabeled nucleotides (or using only unlabeled nucleotides), which can further accelerate the rate of primer extension. Extension of the primer through the intervening region may alternatively or additionally be accelerated by using a mixture of at least two different types of nucleotides in at least one step of the flow order used during extension of the primer through the intervening region. For example, two different bases, such as G and C, may be used simultaneously in the same step, which extends the primer if a complementary C or G base are present. This accelerates extension of the primer by incorporating consecutive bases into the primer even if those bases are of different base types. In some instances, at least one step of the flow order includes 2 different bases. In some instances, at least one step of the flow order includes 3 different bases.

By way of example of extension acceleration, consider a sequence of SEQ ID NO: 1 and the corresponding flow order and flowgram shown in Table 2. The flow order process for extending the sequencing primer hybridized to a polynucleotide containing SEQ ID NO: 1 includes 5 cycles, with Cycles 1, 4, and 5 being the same as each other and Cycles 2 and 3 being the same as each other (with Cycles 1, 4, and 5 being different from Cycles 2 and 3). In this example, each cycle has 4 steps, with Cycles 1, 4, and 5 include the sequential and independent addition of A-C-T-G nucleotides, with a single base type being added at each cycle step. Cycles 2 and 3 include four cycle steps, wherein Step 1 omits A nucleotides (i.e., includes C, T, and G), Step 2 omits, C nucleotides (i.e., includes A, T, and G), Step 3 omits T nucleotides (i.e., includes A, C, and G), and Step 4 omits G nucleotides (i.e., includes A, C, and T). Because Cycles 2 and 3 include multiple different nucleotide base types simultaneously during primer extension, the primer is extended faster during these Cycles than if only a single base type were to be used at any given step. The flowgram shown in Table 2, for extending the primer against the SEQ ID NO: 1 template using the flow order described above, results in up to 6 bases being added in a single flow step during the accelerated portion of primer extension (e.g., Cycle 3, Step 3).

In contrast, Table 3 shows a flowgram of the same SEQ ID NO: 1 using the A-C-T-G cycles with single nucleotides used at each step (similar to Cycles 1, 4, and 5 in Table 2). The flow order used to extend the primer shown in Table 3 requires 10 four-step cycles to extend the primer through the entirety of the polynucleotide, which is substantially slower than the 5 four-step cycles used to extend the primer through the entirety of the polynucleotide using the flow order provided in Table 2.

TABLE 2

| | Flow Cycle | | | | | | | | | | | | | | | | | | | |
|---|---|---|---|---|---|---|---|---|---|---|---|---|---|---|---|---|---|---|---|---|
| | 1 | | | | 2 | | | | 3 | | | | 4 | | | | 5 | | | |
| Sequencing | 1 | 2 | 3 | 4 | 1 | 2 | 3 | 4 | 1 | 2 | 3 | 4 | 1 | 2 | 3 | 4 | 1 | 2 | 3 | 4 |
| Flow Step | | | | | | | | | | | | | | | | | | | | |
| Flow | A | C | T | G | C/T/ | A/T/ | A/C/ | A/C/ | C/T/G | A/T/ | A/C/G | A/C/T | A | C | T | G | A | C | T | G |
| Base(s) | | | | | G | G | G | T | | G | | | | | | | | | | |
| Number of | 1 | 1 | 1 | 1 | 0 | 2 | 1 | 3 | 4 | 3 | 6 | 2 | 0 | 0 | 0 | 1 | 1 | 1 | 1 | 1 |
| Bases | | | | | | | | | | | | | | | | | | | | |
| Incor- | | | | | | | | | | | | | | | | | | | | |
| porated | | | | | | | | | | | | | | | | | | | | |
| Base(s) | A | C | T | G | — | AA | C | TTA | GGCT | ATA | CGGACG | T | C | — | — | — | A | C | T | G |
| Incor- | | | | | | | | | (SEQ ID | | (SEQ ID | | | | | | | | | |
| porated | | | | | | | | | NO: 2) | | NO: 3) | | | | | | | | | |

Example Flowgram for SEQ ID NO: 1: 3′-TGACTTGAATCCGATATGCCTGCAGCTGAC-5′

TABLE 3

| | Flow Cycle | | | | | | | | | | | | | | | | | | | |
|---|---|---|---|---|---|---|---|---|---|---|---|---|---|---|---|---|---|---|---|---|
| Flow Cycle | 1 | | | | 2 | | | | 3 | | | | 4 | | | | 5 | | | |
| Sequencing | 1 | 2 | 3 | 4 | 1 | 2 | 3 | 4 | 1 | 2 | 3 | 4 | 1 | 2 | 3 | 4 | 1 | 2 | 3 | 4 |
| Flow Step | | | | | | | | | | | | | | | | | | | | |
| Flow Base | A | C | T | G | A | C | T | G | A | C | T | G | A | C | T | G | A | C | T | G |
| Number of | 1 | 1 | 1 | 1 | 2 | 1 | 2 | 0 | 1 | 0 | 0 | 2 | 0 | 1 | 1 | 0 | 1 | 0 | 1 | 0 |
| Bases | | | | | | | | | | | | | | | | | | | | |
| Incorporated | | | | | | | | | | | | | | | | | | | | |
| Bases | A | C | T | G | AA | C | TT | — | A | — | — | CC | - | C | T | — | A | — | T | — |
| Incorporated | | | | | | | | | | | | | | | | | | | | |
| Flow Cycle | 6 | | | | 7 | | | | 8 | | | | 9 | | | | 10 | | | |
| Sequencing | 1 | 2 | 3 | 4 | 1 | 2 | 3 | 4 | 1 | 2 | 3 | 4 | 1 | 2 | 3 | 4 | 1 | 2 | 3 | 4 |
| Flow Step | | | | | | | | | | | | | | | | | | | | |
| Flow Base | A | C | T | G | A | C | T | G | A | C | T | G | A | C | T | G | A | C | T | G |
| Number of | 1 | 1 | 0 | 2 | 1 | 1 | 0 | 1 | 0 | 0 | 1 | 0 | 0 | 1 | 0 | 1 | 1 | 1 | 1 | 1 |
| Bases | | | | | | | | | | | | | | | | | | | | |
| Incorporated | | | | | | | | | | | | | | | | | | | | |
| Base(s) | A | C | — | GG | A | C | — | G | - | - | T | — | — | C | — | G | A | C | T | G |
| Incorporated | | | | | | | | | | | | | | | | | | | | |

Example Flowgram for SEQ ID NO: 1: 3′-TGACTTGAATCCGATATGCCTGCAGCTGAC-5′

In some instances, the sequence of an intervening region between barcode regions may be known prior to sequencing, and in such cases a flow order may be selected such that the sequencing primer may be extended during all or most sequencing flow steps. In cases where the sequence of an intervening region is unknown, the sequencing primer may not be extended during each sequencing flow step when a standard flow cycle is used to extend the sequencing primer. An example of this is illustrated by Table 3, Flow Cycle 2, Sequencing Flow Step 4. Thus, the use of a particular predetermined flow order may result in wasted time, as each sequencing flow step takes time to complete, and such time is wasted if the sequencing primer is not extended in each of the sequencing flow steps. By knowing the sequence of the intervening region, however, the set of dark flow steps used to extend the primer through the intervening region may be configured to extend the primer during each dark flow step (i.e., therefore not wasting the time associated with each flow step). This accelerated extension can be performed using single base-type flows (i.e., flow steps with a single type of nucleotide base) or multiple base-type flows (e.g., flow steps with 2 or 3 different types of nucleotide bases) that are configured to extend the primer during each dark flow step. These flow steps need not be executed in a cycle, but can be based on the sequence of the intervening region.

By way of example, consider the sequence of SEQ ID NO: 1 and the corresponding flow order and example flowgram shown in Table 4, for which the primer can be extended through the known sequence of the intervening region in 26 flow steps, where each step comprises a single type of nucleotide base and results in extension of the primer hybridized to the polynucleotide. This flow order performs the primer extension through the known sequence much more quickly (i.e., in 26 sequencing flow steps) than the 40 flow steps that would be required using a predetermined A-C-T-G flow cycle (e.g., as illustrated in Table 3). Primer extension can be further accelerated by using flow steps that include two or three different base types per flow step. For example, consider the sequence of SEQ ID NO: 1 and the corresponding flow order and example flowgram shown in Table 5, in which the primer can be extended through the known sequence of the intervening region in only 8 flow steps, thus substantially increasing the rate of sequencing primer extension.

TABLE 4

| | Sequencing Flow Step | | | | | | | | | | | | | | | | | | | | | | | | | |
|---|---|---|---|---|---|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
| Flow | A | C | T | G | A | C | T | A | G | C | T | A | T | A | C | G | A | C | G | T | C | G | A | C | T | G |
| Base | | | | | | | | | | | | | | | | | | | | | | | | | | |
| Number of | 1 | 1 | 1 | 1 | 2 | 1 | 2 | 1 | 2 | 1 | 1 | 1 | 1 | 1 | 1 | 2 | 1 | 1 | 1 | 1 | 1 | 1 | 1 | 1 | 1 | 1 |
| Bases | | | | | | | | | | | | | | | | | | | | | | | | | | |
| Incorporated | | | | | | | | | | | | | | | | | | | | | | | | | | |
| Base(s) | A | C | T | G | AA | C | TT | A | GG | C | T | A | T | A | C | GG | A | C | G | T | C | G | A | C | T | G |
| Incorporated | | | | | | | | | | | | | | | | | | | | | | | | | | |

Example Flowgram for SEQ ID NO: 1: 3′-TGACTTGAATCCGATATGCCTGCAGCTGAC-5′

TABLE 5

| | Sequencing Flow Step | | | | | | | |
|---|---|---|---|---|---|---|---|---|
| | 1 | 2 | 3 | 4 | 5 | 6 | 7 | 8 |
| Flow | A/C/T | G/A/C | T/A/G | C/T/A | G/A/C | T/C/G | A/C/T | G |
| Base(s) | | | | | | | | |
| Number of | 3 | 4 | 5 | 6 | 5 | 3 | 3 | 1 |
| Bases | | | | | | | | |
| Incorporated | | | | | | | | |
| Base(s) | ACT | GAAC | TTAGG | CTATAC | GGACG | TCG | ACT | G |
| Incorporated | | | | | | | | |

Example Flowgram for SEQ ID NO: 1: 3′-TGACTTGAATCCGATATGCCTGCAGCTGAC-5′

In some embodiments, a method of determining a sequence of a polynucleotide comprising two or more barcode regions on the same end of the polynucleotide relative to a sequence of interest, includes hybridizing a primer to the polynucleotide to form a hybrid, wherein the polynucleotide comprises a first barcode region, a second barcode region, and an intervening region between the first barcode region and the second barcode region; sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark flow steps comprising combining the hybrid with nucleotides without detecting the presence or absence of an incorporated nucleotide; and sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide. The method may further include sequencing the sequence of interest, optionally after the two or more barcode regions are sequenced. Optionally, the polynucleotide further includes a unique molecular identifier (UMI).

In some embodiments, a method of determining a sequence of a polynucleotide comprising two or more barcode regions on the same end of the polynucleotide relative to a sequence of interest, includes hybridizing a primer to the polynucleotide to form a hybrid, wherein the polynucleotide comprises a first barcode region, a second barcode region, and an intervening region with a known sequence between the first barcode region and the second barcode region; sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark flow steps comprising combining the hybrid with nucleotides without detecting the presence or absence of an incorporated nucleotide, wherein the set of one or more dark flow steps used to extend the primer through the intervening region is configured to extend the primer during each dark flow step; and sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide. The method may further include sequencing the sequence of interest, optionally after the two or more barcode regions are sequenced. Optionally, the polynucleotide further includes a unique molecular identifier (UMI).

In some embodiments, a method of determining a sequence of a polynucleotide comprising three or more barcode regions on the same end of the polynucleotide relative to a sequence of interest, includes hybridizing a primer to the polynucleotide to form a hybrid, wherein the polynucleotide comprises a first barcode region, a second barcode region, an intervening region between the first barcode region and the second barcode region, a third barcode region, and a second intervening region between the second barcode region and the third barcode region; sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark flow steps comprising combining the hybrid with nucleotides without detecting the presence or absence of an incorporated nucleotide; sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide; extending the primer through a second intervening region using a second set of one or more dark flow steps comprising combining the hybrid with nucleotides without detecting the presence or absence of an incorporated nucleotide; and sequencing a third barcode region in the polynucleotide using a third plurality of sequencing flow steps comprising combining the hybrid with labeled nucleotides and detecting the presence or absence of an incorporated nucleotide. The method may further include sequencing the sequence of interest, optionally after the two or more barcode regions are sequenced. Optionally, the polynucleotide further includes a unique molecular identifier (UMI).

In some embodiments, a method of determining a sequence of a polynucleotide comprising three or more barcode regions on the same end of the polynucleotide relative to a sequence of interest, includes hybridizing a primer to the polynucleotide to form a hybrid, wherein the polynucleotide comprises a first barcode region, a second barcode region, an intervening region with a known sequence between the first barcode region and the second barcode region, a third barcode region, and a second intervening region with a known sequence between the second barcode region and the third barcode region; sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide; extending the primer through the intervening region using a set of one or more dark flow steps comprising combining the hybrid with nucleotides without detecting the presence or absence of an incorporated nucleotide, wherein the set of one or more dark flow steps used to extend the primer through the first intervening region is configured to extend the primer during each dark flow step; sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide; extending the primer through a second intervening region using a second set of one or more dark flow steps comprising combining the hybrid with nucleotides without detecting the presence or absence of an incorporated nucleotide, wherein the set of one or more dark flow steps used to extend the primer through the second intervening region is configured to extend the primer during each dark flow step; and sequencing a third barcode region in the polynucleotide using a third plurality of sequencing flow steps comprising combining the hybrid with labeled nucleotides and detecting the presence or absence of an incorporated nucleotide. The method may further include sequencing the sequence of interest, optionally after the two or more barcode regions are sequenced. Optionally, the polynucleotide further includes a unique molecular identifier (UMI).

The methods described herein optionally further include reporting information determined using the analytical methods and/or generating a report containing the information determined suing the analytical methods. For example, in some embodiments, the method further includes reporting or generating a report containing related to the identification of a variant in a polynucleotide derived from a subject (e.g., within a subject's genome). Reported information or information within the report may be associated with, for example, a locus of a coupled sequencing read pair mapped to a reference sequence, a detected variant (such as a detected structural variant or detected SNP), one or more assembled consensus sequences and/or a validation statistic for the one or more assembled consensus sequences. The report may be distributed to or the information may be reported to a recipient, for example a clinician, the subject, or a researcher.

### Exemplary Embodiments

The following embodiments are exemplary and are not intended to limit the scope of the claimed invention.

Embodiment 1. A method of determining a sequence of a polynucleotide comprising two or more barcode regions on the same end of the polynucleotide relative to a region of interest, comprising:
hybridizing a primer to the polynucleotide to form a hybrid, wherein the polynucleotide comprises a first barcode region, a second barcode region, and an intervening region between the first barcode region and the second barcode region;
sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide;
extending the primer through the intervening region using a set of one or more dark sequencing flow steps comprising combining the hybrid with nucleotides without detecting the presence or absence of an incorporated nucleotide; and
sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide.

Embodiment 2. The method of embodiment 1, wherein the intervening region has a known sequence.

Embodiment 3. The method of embodiment 2, wherein the set of one or more dark sequencing flow steps used to extend the primer through the intervening region is configured to extend the primer during each dark sequencing flow step.

Embodiment 4. The method of any one of embodiments 1-3, wherein the intervening region is formed by ligation or PCR amplification.

Embodiment 5. The method of any one of embodiments 1-4, further comprising sequencing the region of interest in the polynucleotide.

Embodiment 6. The method of embodiment 5, wherein the region of interest is sequenced after sequencing the first barcode region and the second barcode region.

Embodiment 7. The method of any one of embodiments 1-6, further comprising:
extending the primer through a second intervening region using a second set of one or more dark flow steps comprising combining the hybrid with nucleotides without detecting the presence or absence of an incorporated nucleotide; and
sequencing a third barcode region in the polynucleotide using a third plurality of sequencing flow steps comprising combining the hybrid with labeled nucleotides and detecting the presence or absence of an incorporated nucleotide;
wherein the second intervening region is between the second barcode region and the third barcode region, and wherein the third barcode region is on the same end of the polynucleotide relative to the region of interest as the first barcode region and the second barcode region.

Embodiment 8. The method of embodiment 7, wherein the region of interest is sequenced after sequencing the first barcode region, the second barcode region, and the third barcode region.

Embodiment 9. The method of embodiment 7 or 8, wherein the second intervening region has a known sequence.

Embodiment 10. The method of embodiment 9, wherein the second set of one or more dark sequencing flow steps used to extend the primer through the second intervening region is configured to extend the primer during each dark flow step.

Embodiment 11. The method of any one of embodiments 7-10, wherein the second intervening region is formed by ligation or PCR amplification.

Embodiment 12. The method of any one of embodiments 1-11, further comprising associating the two or more barcode regions with the region of interest.

Embodiment 13. The method of any one of embodiments 1-12, wherein the nucleotides used in the first plurality of sequencing flow steps or the nucleotides used in the second plurality of sequencing flow steps comprise non-terminating nucleotides.

Embodiment 14. The method of any one of embodiments 1-13, wherein the nucleotides used in the set of one or more dark sequencing flow steps comprise non-terminating nucleotides.

Embodiment 15. The method of any one of embodiments 1-14, wherein the nucleotides used in the first plurality of sequencing flow steps or the nucleotides used in the second plurality of sequencing flow steps comprise labeled nucleotides and unlabeled nucleotides.

Embodiment 16. The method of any one of embodiments 1-15, wherein the nucleotides used in the dark sequencing flow steps comprise unlabeled nucleotides.

Embodiment 17. The method of any one of embodiments 1-16, wherein the nucleotides used in the dark sequencing flow steps comprise only unlabeled nucleotides.

Embodiment 18. The method of any one of embodiment 1-17, wherein the polynucleotide further comprises a unique molecular identifier.

Embodiment 19. The method of embodiment 18, wherein the unique molecular identifier is directly fused to one of the two or more barcode regions.

Embodiment 20. The method of any one of embodiments 1-19, wherein the polynucleotide is a cDNA molecule.

Embodiment 21. The method of any one of embodiments 1-20, wherein the nucleotides used in each sequencing flow step of the first plurality of sequencing flow steps comprise a single type of nucleotide base.

Embodiment 22. The method of any one of embodiments 1-21, wherein the nucleotides used in at least a portion of the sequencing flow steps of the first plurality of sequencing flow steps comprise two or three different types of nucleotide bases.

Embodiment 23. The method of any one of embodiments 1-22, wherein the nucleotides used in each sequencing flow step of the second plurality of sequencing flow steps comprise a single type of nucleotide base.

Embodiment 24. The method of any one of embodiments 1-23, wherein the nucleotides used in at least a portion of the sequencing flow steps of the second plurality of sequencing flow steps comprise two or three different types of nucleotide bases.

Embodiment 25. The method of any one of embodiments 1-24, wherein the nucleotides used in each dark sequencing flow step comprises a single type of nucleotide base.

Embodiment 26. The method of any one of embodiments 1-25, wherein the nucleotides used in at least a portion of the dark sequencing flow steps comprise two or three different types of nucleotide bases.

Embodiment 27. The method of any one of embodiments 1-26, wherein the two or more barcode regions, in combination, uniquely identify a cell of origin for the polynucleotide.

Embodiment 28. The method of any one of embodiments 1-27, further comprising labeling a nucleic acid molecule from a cell with a cell-specific combination of the two or more barcode regions to form the polynucleotide.

Embodiment 29. The method of embodiment 28, wherein the two or more barcode regions comprise three or more barcode regions on the same end of the polynucleotide relative to the region of interest, each barcode region separated from an adjacent barcode region by an intervening region.

Embodiment 30. The method of any one of embodiments 28 or 29, wherein labeling the nucleic acid molecule comprises ligating the at least one of the two or more barcode regions to the nucleic acid molecule.

Embodiment 31. The method of embodiment 30, wherein labeling the nucleic acid molecule comprises ligating the at least one of the two or more barcode regions to the nucleic acid molecule within a cell.

Embodiment 32. The method of any one of embodiments 28-31, wherein labeling the nucleic acid molecule comprises reverse transcribing an mRNA molecule to form a cDNA molecule comprising one of the two or more barcode regions.

Embodiment 33. The method of embodiment 32, wherein the mRNA molecule is reverse transcribed within the cell.

Embodiment 34. The method of any one of embodiments 28-33, wherein labeling the nucleic acid molecule comprises attaching at least one of the two or more barcode regions to the polynucleotide by polymerase chain reaction (PCR) amplification.

Embodiment 35. The method of any one of embodiments 28-34, wherein labeling the nucleic acid molecule comprises attaching at least one of the two or more barcode regions to the polynucleotide by polymerase chain reaction (PCR) amplification outside of the cell.

Embodiment 36. The method of any one of embodiments 1-35, wherein a sequence of each of a plurality of polynucleotides having different sequences is determined in parallel.

Embodiment 37. The method of embodiment 36, wherein at least a portion the polynucleotides in the plurality of polynucleotides having different sequences have the same two or more barcode regions.

Embodiment 38. The method of embodiment 37, wherein polynucleotides having the same two or more barcode regions are associated with the same cell of origin.

### EXAMPLES

### Example 1: Sequencing Polynucleotides, where Each Polynucleotide Comprises a First Barcode, a Linker Region, a Second Barcode, and a Region of Interest

As described herein, polynucleotides may comprise multiple barcode sequences that can in combination be used to uniquely label each polynucleotide in a plurality of polynucleotides. A set of barcode sequences may be selected such that each barcode sequence is distinct and that each barcode sequence in the set may be analyzed (e.g., the sequence of each barcode may be determined) within a predetermined number of sequence flows. Based at least in part on the predetermined number of flows (e.g., 3, 4, 5, 6, etc. flows), and a predetermined nucleotide base type flow order a large variety of distinct barcode sequences are available (e.g., at least 100, at least 1000, at least 10,000).

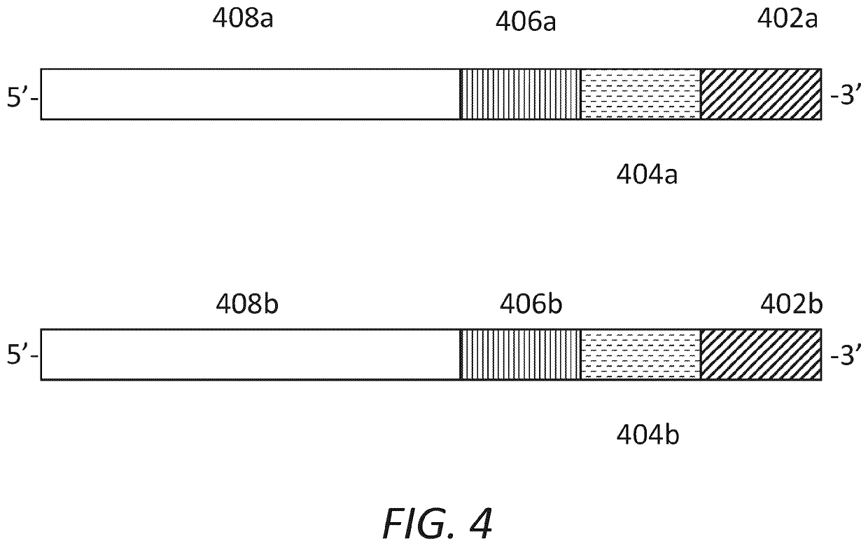

A) Sequencing barcodes in a first set of barcodes. Tables 6 and 7 illustrate flowgrams for 2 distinct barcode sequences (i.e., SEQ ID NO: 4 and SEQ ID NO: 5) that may each be analyzed within four flow cycles using the predetermined flow order of T-G-C-A. SEQ ID NO: 4 comprises 9 bases, while SEQ ID NO: 5 comprises 11 bases. In other words, although these two example barcode sequences comprise different numbers of bases (i.e., different lengths in base space), they each require the same number of flows (i.e., they have the same length in flow space) in order to be sequenced according to sequencing methods described herein (e.g., flow sequencing). In this example, SEQ ID NOs 4 and 5 represent first barcode sequences, each first barcode sequence being attached (e.g., covalently bound via ligation) to a sequence of interest (e.g., a region of interest) of a respective polynucleotide. Here, each barcode is located 3′ to a corresponding sequence of interest in a respective polynucleotide. In FIG. 4, regions 402 correspond to first barcodes (e.g., 402a illustrates SEQ ID NO: 4 and 402b illustrates SEQ ID NO:5).

TABLE 6

| Flow Cycle | 1 | | | | 2 | | | | 3 | | | | 4 | | | |
|---|---|---|---|---|---|---|---|---|---|---|---|---|---|---|---|---|
| Sequencing | 1 | 2 | 3 | 4 | 1 | 2 | 3 | 4 | 1 | 2 | 3 | 4 | 1 | 2 | 3 | 4 |
| Flow Step | | | | | | | | | | | | | | | | |
| Flow Base(s) | T | G | C | A | T | G | C | A | T | G | C | A | T | G | C | A |
| Number of Bases | 0 | 1 | 0 | 0 | 1 | 1 | 1 | 1 | 1 | 0 | 1 | 0 | 1 | 1 | 0 | 0 |
| Incorporated | | | | | | | | | | | | | | | | |

Flowgram for SEQ ID NO: 4: 3'-GTGCATCTG-5'

TABLE 7

| Flow Cycle | 1 | | | | 2 | | | | 3 | | | | 4 | | | |
|---|---|---|---|---|---|---|---|---|---|---|---|---|---|---|---|---|
| Sequencing | 1 | 2 | 3 | 4 | 1 | 2 | 3 | 4 | 1 | 2 | 3 | 4 | 1 | 2 | 3 | 4 |
| Flow Step | | | | | | | | | | | | | | | | |
| Flow Base(s) | T | G | C | A | T | G | C | A | T | G | C | A | T | G | C | A |
| Number of Bases | 1 | 0 | 2 | 0 | 2 | 0 | 1 | 1 | 1 | 0 | 1 | 0 | 0 | 1 | 1 | 0 |
| Incorporated | | | | | | | | | | | | | | | | |

Flowgram for SEQ ID NO: 5: 3′-TCCTTCATCGC-5'

In each polynucleotide (e.g., polynucleotides 410a and 410b as illustrated in FIG. 4), the respective first barcode 402 is disposed 3′ to an intervening region 404). Thus, sequencing primers will first be extended through the first barcodes (e.g., using the predetermined number of flow cycles as shown in Tables 6 and 7). Next, sequencing primers will be extended through intervening regions 404.

As described elsewhere herein, intervening regions may be traversed (e.g., sequencing primers may be extended through intervening regions) by using flow cycles including two or more nucleotide base types (e.g., at a faster rate of addition of nucleotides per flow cycle than is possible in flow cycles using single nucleotide base types). In this example, sequencing primers are extended through intervening regions 404a and 404b by using 2 flow cycles of C, G, and A nucleotides (e.g., the flows are lacking in T nucleotides). The sequencing of the intervening regions will include G, C, and T nucleotides. Thus, when an A nucleotide in the polynucleotide is encountered by a polymerase, extension will stall until T nucleotides are introduced in another flow cycle.

Additional barcodes (e.g., second barcodes 406a and 406b) will be sequenced after intervening regions 404 to polynucleotides, as described herein. These second barcodes will all begin with an A nucleotide base (e.g., the first flow that will enable a sequencing primer to be extended into the second barcodes will be a sequencing flow step including T nucleotides). Examples of two distinct second barcodes are SEQ ID NO: 6 and SEQ ID NO: 7. In this instance, each second barcode may each be analyzed within five flow cycles using the predetermined flow order of T-G-C-A. Tables 6 and 7 illustrate respective flowgrams SEQ ID NO: 6 and SEQ ID NO: 7. SEQ ID NO: 6 comprises 15 bases, while SEQ ID NO: 7 comprises 9 bases. In other words, as with first barcodes 402 described above, although these second barcode sequences comprise different numbers of bases (i.e., have different lengths in base space), they each require the same number of flows in order to be sequenced according to flow sequencing methods described herein (i.e., they have the same length in flow space).

TABLE 6

| Flow Cycle | 1 | | | | 2 | | | | 3 | | | | 4 | | | | 5 | | | |
|---|---|---|---|---|---|---|---|---|---|---|---|---|---|---|---|---|---|---|---|---|
| Sequencing | 1 | 2 | 3 | 4 | 1 | 2 | 3 | 4 | 1 | 2 | 3 | 4 | 1 | 2 | 3 | 4 | 1 | 2 | 3 | 4 |
| Flow Step | | | | | | | | | | | | | | | | | | | | |
| Flow Base(s) | T | G | C | A | T | G | C | A | T | G | C | A | T | G | C | A | T | G | C | A |
| Number of Bases | 1 | 0 | 2 | 0 | 1 | 1 | 1 | 0 | 0 | 1 | 1 | 1 | 0 | 3 | 0 | 0 | 0 | 0 | 0 | 1 |
| Incorporated | | | | | | | | | | | | | | | | | | | | |

Flowgram for SEQ ID NO: 6: 3'-TCCTGTGCGCAGGGA-5'

TABLE 7

| Flow Cycle | 1 | | | | 2 | | | | 3 | | | | 4 | | | | 5 | | | |
|---|---|---|---|---|---|---|---|---|---|---|---|---|---|---|---|---|---|---|---|---|
| Sequencing | 1 | 2 | 3 | 4 | 1 | 2 | 3 | 4 | 1 | 2 | 3 | 4 | 1 | 2 | 3 | 4 | 1 | 2 | 3 | 4 |
| Flow Step | | | | | | | | | | | | | | | | | | | | |
| Flow Base(s) | T | G | C | A | T | G | C | A | T | G | C | A | T | G | C | A | T | G | C | A |
| Number of Bases | 1 | 1 | 1 | 1 | 0 | 0 | 1 | 0 | 0 | 1 | 0 | 0 | 1 | 0 | 0 | 1 | 1 | 0 | 0 | 0 |
| Incorporated | | | | | | | | | | | | | | | | | | | | |

Flowgram for SEQ ID NO: 7: 3'-TGCACGTAT-5'

Thus, polynucleotides 410 organized as illustrated in FIG. 4 may be identified with barcode regions sequenced determined according to the flow cycles described above.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A method of determining a sequence of a polynucleotide comprising two or more barcode regions on the same end of the polynucleotide relative to a region of interest, comprising:
hybridizing a primer to the polynucleotide to form a hybrid, wherein the polynucleotide comprises a first barcode region, a second barcode region, and an intervening region between the first barcode region and the second barcode region;
sequencing the first barcode region in the polynucleotide using a first plurality of sequencing flow steps, each sequencing flow step in the first plurality of sequencing flow steps comprising combining the hybrid with nucleotides, wherein at least a portion of the nucleotides are labeled, and detecting the presence or absence of an incorporated nucleotide;
extending the primer through the intervening region using a set of one or more dark sequencing flow steps comprising combining the hybrid with nucleotides without detecting the presence or absence of an incorporated nucleotide; and
sequencing the second barcode region in the polynucleotide using a second plurality of sequencing flow steps, each sequencing flow step in the second plurality of sequencing flow steps comprising combining the hybrid with nucleotides, wherein at least a portion of the nucleotides are labeled, and detecting the presence or absence of an incorporated nucleotide.

2. The method of claim 1, wherein the intervening region has a known sequence.

3. The method of claim 2, wherein the set of one or more dark sequencing flow steps used to extend the primer through the intervening region is configured to extend the primer during each dark sequencing flow step.

4. The method of any one of claims 1-3, wherein the intervening region is formed by ligation or PCR amplification.

5. The method of any one of claims 1-4, further comprising sequencing the region of interest in the polynucleotide.

6. The method of claim 5, wherein the region of interest is sequenced after sequencing the first barcode region and the second barcode region.

7. The method of any one of claims 1-6, further comprising
extending the primer through a second intervening region using a second set of one or more dark flow steps comprising combining the hybrid with nucleotides without detecting the presence or absence of an incorporated nucleotide; and
sequencing a third barcode region in the polynucleotide using a third plurality of sequencing flow steps comprising combining the hybrid with labeled nucleotides and detecting the presence or absence of an incorporated nucleotide;
wherein the second intervening region is between the second barcode region and the third barcode region, and wherein the third barcode region is on the same end of the polynucleotide relative to the region of interest as the first barcode region and the second barcode region.

8. The method of claim 7, wherein the region of interest is sequenced after sequencing the first barcode region, the second barcode region, and the third barcode region.

9. The method of claim 7 or 8, wherein the second intervening region has a known sequence.

10. The method of claim 9, wherein the second set of one or more dark flow steps used to extend the primer through the second intervening region is configured to extend the primer during each dark sequencing flow step.

11. The method of any one of claims 7-10, wherein the second intervening region is formed by ligation or PCR amplification.

12. The method of any one of claims 1-11, further comprising associating the two or more barcode regions with the region of interest.

13. The method of any one of claims 1-12, wherein the nucleotides used in the first plurality of sequencing flow steps or the nucleotides used in the second plurality of sequencing flow steps comprise non-terminating nucleotides.

14. The method of any one of claims 1-13, wherein the nucleotides used in the set of one or more dark sequencing flow steps comprise non-terminating nucleotides.

15. The method of any one of claims 1-14, wherein the nucleotides used in the first plurality of sequencing flow steps or the nucleotides used in the second plurality of sequencing flow steps comprise labeled nucleotides and unlabeled nucleotides.

16. The method of any one of claims 1-15, wherein the nucleotides used in the dark sequencing flow steps comprise unlabeled nucleotides.

17. The method of any one of claims 1-16, wherein the nucleotides used in the dark sequencing flow steps comprise only unlabeled nucleotides.

18. The method of any one of claims 1-17, wherein the polynucleotide further comprises a unique molecular identifier.

19. The method of claim 18, wherein the unique molecular identifier is directly fused to one of the two or more barcode regions.

20. The method of any one of claims 1-19, wherein the polynucleotide is a cDNA molecule.

21. The method of any one of claims 1-20, wherein the nucleotides used in each sequencing flow step of the first plurality of sequencing flow steps comprise a single type of nucleotide base.

22. The method of any one of claims 1-21, wherein the nucleotides used in at least a portion of the sequencing flow steps of the first plurality of sequencing flow steps comprise two or three different types of nucleotide bases.

23. The method of any one of claims 1-22, wherein the nucleotides used in each sequencing flow step of the second plurality of sequencing flow steps comprise a single type of nucleotide base.

24. The method of any one of claims 1-23, wherein the nucleotides used in at least a portion of the sequencing flow steps of the second plurality of sequencing flow steps comprise two or three different types of nucleotide bases.

25. The method of any one of claims 1-24, wherein the nucleotides used in each dark sequencing flow step comprises a single type of nucleotide base.

26. The method of any one of claims 1-25, wherein the nucleotides used in at least a portion of the dark sequencing flow steps comprise two or three different types of nucleotide bases.

27. The method of any one of claims 1-26, wherein the two or more barcode regions, in combination, uniquely identify a cell of origin for the polynucleotide.

28. The method of any one of claims 1-27, further comprising labeling a nucleic acid molecule from a cell with a cell-specific combination of the two or more barcode regions to form the polynucleotide.

29. The method of claim 28, wherein the two or more barcode regions comprise three or more barcode regions on the same end of the polynucleotide relative to the region of interest, each barcode region separated from an adjacent barcode region by an intervening region.

30. The method of any one of claim 28 or 29, wherein labeling the nucleic acid molecule comprises ligating the at least one of the two or more barcode regions to the nucleic acid molecule.

31. The method of claim 30, wherein labeling the nucleic acid molecule comprises ligating the at least one of the two or more barcode regions to the nucleic acid molecule within a cell.

32. The method of any one of claims 28-31, wherein labeling the nucleic acid molecule comprises reverse transcribing an mRNA molecule to form a cDNA molecule comprising one of the two or more barcode regions.

33. The method of claim 32, wherein the mRNA molecule is reverse transcribed within the cell.

34. The method of any one of claims 28-33, wherein labeling the nucleic acid molecule comprises attaching at least one of the two or more barcode regions to the polynucleotide by polymerase chain reaction (PCR) amplification.

35. The method of any one of claims 28-34, wherein labeling the nucleic acid molecule comprises attaching at least one of the two or more barcode regions to the polynucleotide by polymerase chain reaction (PCR) amplification outside of the cell.

36. The method of any one of claims 1-35, wherein a sequence of each of a plurality of polynucleotides having different sequences is determined in parallel.

37. The method of claim 36, wherein at least a portion the polynucleotides in the plurality of polynucleotides having different sequences have the same two or more barcode regions.

38. The method of claim 37, wherein polynucleotides having the same two or more barcode regions are associated with the same cell of origin.


SEQUENCE LISTING (7 sequences)

SEQ ID NO: 1 - DNA, 30 bp, Artificial Sequence
  Synthetic Construct
cagtcgacgtccgtatagcctaagttcagt

SEQ ID NO: 2 - DNA, 4 bp, Artificial Sequence
  Synthetic Construct
ggct

SEQ ID NO: 3 - DNA, 6 bp, Artificial Sequence
  Synthetic Construct
cggacg

SEQ ID NO: 4 - DNA, 9 bp, Artificial Sequence
  Synthetic Construct
gtctacgtg

SEQ ID NO: 5 - DNA, 11 bp, Artificial Sequence
  Synthetic Construct
cgctacttcct

SEQ ID NO: 6 - DNA, 15 bp, Artificial Sequence
  Synthetic Construct
agggacgcgtgtcct

SEQ ID NO: 7 - DNA, 9 bp, Artificial Sequence
  Synthetic Construct
tatgcacgt